(19) United States
(12) Patent Application Publication
(10) Pub. No.: US 2025/0262342 A1
(43) Pub. Date: Aug. 21, 2025

(54) CONTACT LENS PACKAGING SOLUTION AND CONTACT LENS PRODUCT COMPRISING THE SAME

(71) Applicant: BenQ Materials Corporation, Taoyuan (TW)

(72) Inventors: Li-Pin Lin, Taoyuan (TW); Hsia-Hao Chang, Taoyuan (TW); Fan-Dan Jan, Taoyuan (TW)

(73) Assignee: BenQ Materials Corporation, Taoyuan (TW)

(21) Appl. No.: 18/804,290

(22) Filed: Aug. 14, 2024

(30) Foreign Application Priority Data
Feb. 15, 2024 (TW) 113105237

(51) Int. Cl. A61L 12/14; G02C 7/04

(52) U.S. Cl. CPC A61L 12/14; G02C 7/04

(57) ABSTRACT

The invention is related to a contact lens packaging solution and a contact lens product comprising the same, wherein the contact lens packaging solution comprises 0.1 wt % to 0.4 wt % of a moisturizing agent and a buffer solution, wherein the moisturizing agent comprises a salt of hyaluronic acid and a hydrolyzed hyaluronic acid derivative and the weight ratio of the hyaluronic acid and the hydrolyzed hyaluronic acid derivative is in the range of 1:4 to 4:1, and the contact lens product comprises a contact lens packaging solution and a contact lens soaked therein. The contact lens packaging solution of the present invention can provide the contact lens wearer with satisfied initial comfort.

### CROSS-REFERENCE TO RELATED APPLICATION

This application claims the benefit of Taiwanese patent application serial No. TW113105237, filed on Feb. 15, 2024, the subject matters of which are incorporated herein by reference.

### BACKGROUND OF THE INVENTION

### Field of Invention

This invention relates to a contact lens packaging solution and a contact lens product, and particularly relates to a contact lens packaging solution for enhancing the initial comfort of the contact lens and a contact lens product comprising a contact lens packaging solution and a contact lens immersed therein.

### Description of Related Art

Soft contact lenses are individually packed in plastic blisters containing buffer solution for transportation and sales. The buffer solution in the plastic blister commonly is saline solution to make the contact lenses be worn straight from the blister pack. Therefore, the pH value and the osmolality of the buffer solution should be similar to the eye tear to avoid discomfort caused by the initial wearing. For improving the wearer comfort, the buffer solution for packaging usually further contains nonionic surfactant, surface lubricant and moisture agent to provide the wearer comfort.

The well-known moisture agents suitably used for soft contact lens packaging solution are, for example, poly(meth)acrylamide, polyhydroxyalkyl (meth)acrylate, polyvinyl pyrrolidone, hyaluronic acid, xanthan gum, arabic gum and starch, wherein the hyaluronic acid, a natural polysaccharide with biocompatiblity, absorbability, moisture and lubrication, has been widely used in ophthalmic preparations. However, because of the high viscosity of the hyaluronic acid, the increased amount of hyaluronic acid for increasing the moisture of the packaging solution will increase the viscosity thereof. When using the packaging solution with high content of hyaluronic acid, the water layer formed by the high viscous packaging solution on the contact lens tends to adsorb lipid to affect the comfort thereof. Recently, it has been suggested to use the hydrolyzed hyaluronic acid derivative in the multi-purpose contact lens solution and eye drops. The hydrolyzed hyaluronic acid derivative can impart the hydrophilicity to the surface of the contact lenses and inhibit the adsorption of the cation antimicrobial agent to surface of the contact lens. However, because the sterilization of the contact lens is conducted at high temperature and under high pressure after packaging with packaging solution, the hydrolyzed hyaluronic acid derivative with small molecular weight will be hydrolyzed at the high temperature to make the pH of the packaging solution decrease and the color appearance thereof be yellow. In such a situation, if the contact lenses are taken out of the packaging and put on them directly, it will cause discomfort to the wearer.

Therefore, the present invention discloses a contact lens packaging solution for contact lens, which can provide wearers with sufficient initial comfort after the contact lens put in the eyes directly from the packaging solution, enhance the hydration of the contact lens to slow down the dryness of the contact lens, reduce the eye fatigue caused by long time wearing and improve the wear comfort of the contact lens.

### SUMMARY OF THE INVENTION

The present invention is to provide a packaging solution for contact lenses to provide the hydration of the contact lens and alleviate the eye dryness caused by wearing contact lenses.

The contact lens packaging solution disclosed in the present invention comprises a buffer solution and 0.1 wt % to 0.4 wt % of a moisturizing agent, wherein the moisturizing agent comprises a salt of hyaluronic acid and a hydrolyzed hyaluronic acid derivative, and the weight ratio of the salt of hyaluronic acid and the hydrolyzed hyaluronic acid derivative is in the range of 1:4 to 4:1.

The pH value of the disclosed contact lens packaging solution is in the range of 6.8 to 7.5.

In the disclosed contact lens packaging solution, the salt of hyaluronic acid in the moisturizing agent is a sodium hyaluronate with a molecular weight in the range of 850,000 to 2,200,000.

In the disclosed contact lens packaging solution, the hydrolyzed hyaluronic acid derivative in the moisturizing agent is an alkyl glyceryl hydrolyzed hyaluronate having a molecular weight less than 10,000 and having a chemical structure of the following formula (I):

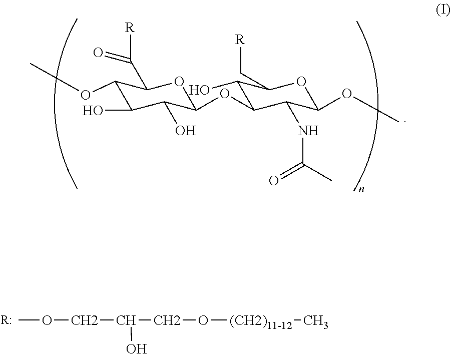

The buffer solution for the contact lens packaging solution of the present invention can be 0.1 wt % to 3 wt % of a borate buffer solution or a phosphate buffer solution.

In an embodiment of the present invention, the contact lens packaging solution can further comprise 0.005 wt % to 0.05 wt % of an alginic acid.

The buffer solution for the contact lens packaging solution of the present invention is a borate buffer solution comprising sodium chloride, boric acid and sodium borate.

In an embodiment of the present invention, the contact lens packaging solution can further comprise an antimicrobial agent, a thickener, a lubricant or a combination thereof.

In another aspect, the present invention is to provide a contact lens product comprising a packaging solution and a contact lens immersed in packaging solution, wherein the packaging solution comprises a buffer solution and 0.1 wt % to 0.4 wt % of a moisturizing agent, wherein the moisturizing agent comprises a salt of hyaluronic acid and a hydrolyzed hyaluronic acid derivative, and the weight ratio of the salt of hyaluronic acid and the hydrolyzed hyaluronic acid derivative is in the range of 1:4 to 4:1.

In the contact lens product of the present invention, the contact lens can be hydrogel contact lenses or silicone hydrogel contact lenses.

### DETAILED DESCRIPTION OF THE INVENTION

In order to make the above and other objects, features, and advantages of the present invention more obvious and easy to understand, preferred Examples are listed below and are described in detail below with the attached chemical formula.

The present invention is to provide a contact lens packaging solution comprising a buffer solution and a moisturizing agent, wherein the moisturizing agent comprises a salt of hyaluronic acid and a hydrolyzed hyaluronic acid derivative, to provide the hydration of the contact lenses for sufficient initial comfort and improve the eye dryness caused by wearing contact lenses.

The contact lens packaging solution disclosed in the present invention comprises a buffer solution and 0.1 wt % to 0.4 wt % of a moisturizing agent, wherein the moisturizing agent comprises a salt of hyaluronic acid and a hydrolyzed hyaluronic acid derivative and the weight ratio of the salt of hyaluronic acid, and the hydrolyzed hyaluronic acid derivative is in the range of 1:4 to 4:1. In a preferred embodiment of the present invention, the contact lens packaging solution comprises a buffer solution and 0.2 wt % to 0.3 wt % of a moisturizing agent, wherein the moisturizing agent comprises a salt of hyaluronic acid and a hydrolyzed hyaluronic acid derivative, and the weight ratio of the salt of hyaluronic acid and the hydrolyzed hyaluronic acid derivative is in the range of 1:2 to 3:1.

Because soft contact lenses can be put on the eyes directly after taken out from the packaging container, the packaging solution in the container will be adsorbed on the contact lenses. Therefore, the pH value and osmolality of the contact lens packaging solution close to those of tear will improve the eye comfort. The initial pH value of the contact lens packaging solution of the present invention and the pH value after sterilization thereof are both in the range of 6.8 to 7.5 and preferably in the range of 6.9 to 7.3. The moisturizing agent of the contact lens packaging solution of the present invention comprises a salt of hyaluronic acid and a hydrolyzed hyaluronic acid derivative, which keeps the pH value of the packaging solution in the range of 6.8 to 7.5 and the osmolality thereof greater than 300 mOsmol/Kg and relieve the decrease of the pH value caused by the hydrolysis of the hydrolyzed hyaluronic acid derivative or the color appearance change during the high-temperature sterilization process. The wear discomfort may occur when the packaging solution with a lower pH value is adsorbed by the contact lenses.

In the disclosed contact lens packaging solution, the salt of hyaluronic acid in the moisturizing agent is a sodium hyaluronate with a molecular weight in the range of 850,000 to 2,200,000. The sodium hyaluronate suitably used in the disclosed contact lens packaging solution can be commercial products, such as HA-LQ or HA-LQH from Kewpie Co. Japan.

In the disclosed contact lens packaging solution, the hydrolyzed hyaluronic acid derivative in the moisturizing agent is an alkyl glyceryl hydrolyzed hyaluronate having a molecular weight less than 10,000, and preferably, the alkyl glyceryl hydrolyzed hyaluronate having a chemical structure of the following formula (I):

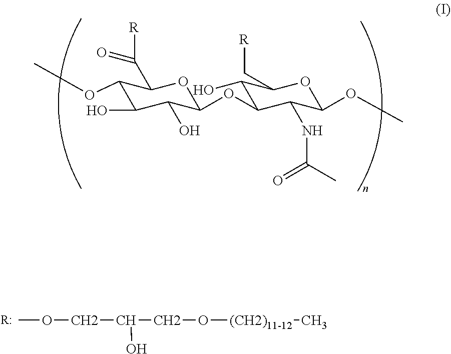

The suitable alkyl glyceryl hydrolyzed hyaluronate used in the present invention can be, such as Halorepair® manufactured by Kewpie Co., Japan.

The contact lens packaging solution of the present invention is suitable for packaging hydrogel contact lenses and silicone hydrogel contact lenses.

The buffer solution used in the contact lens packaging solution of the present invention can be the buffer solution with physiological compatibility, such as borate and boric acid buffer solution, phosphate buffer solution, citrate buffer solution, carbonate buffer solution or acetate buffer solution. The buffer solutions can be borate buffer solution or phosphate buffer solution with alkali metal salt, such as sodium salt or potassium salt in a concentrate of 0.1 wt % to 3 wt %. In another embodiment of the present invention, the buffer solution of the contact lens packaging solution preferably comprises water, sodium chloride, boric acid and borate.

The moisturizing agent contained in the contact lens packaging solution of the present invention provide adequate pH value and osmolality. Furthermore, after the lens is taken out from the package, the water layer remaining on the contact lenses can last for a long time to keep the eye hydration sufficient and the wearer initial comfort.

The contact lens packaging solution of the present invention can further comprise alginic acid as moisturizing aid to enhance the hydration to the contact lenses and alleviate the eye dryness when wearing the lens. In the contact lens packaging solution of the present invention, the weight average molecular weight of the alginic acid suitably used is in the range of 1,000 to 30,000 and preferably in the range of 5,000 to 20,000. The amount of the alginic acid is present in a range of 0.005 wt % to 0.05 wt %.

In the moisturizing agent of the present contact lens packaging solution, the components for preparing the moisturizing agent can be mixed and added into the buffer solution or can be added individually into the buffer solution without any specific limiting.

Furthermore, the present contact lens packaging solution can optionally further comprise antimicrobial agents, thickeners, lubricants or the combinations thereof.

In another aspect, the present invention is to provide a contact lens product comprising a packaging solution and a contact lens immersed in packaging solution, wherein the packaging solution comprises a buffer solution and 0.1 wt % to 0.4 wt % of a moisturizing agent, wherein the moisturizing agent comprises a salt of hyaluronic acid and a hydrolyzed hyaluronic acid derivative, and the weight ratio of the salt of hyaluronic acid and the hydrolyzed hyaluronic acid derivative is in the range of 1:4 to 4:1.

In the contact lens product of the present invention, the contact lens can be hydrogel contact lenses or silicone hydrogel contact lenses. The hydrogel contact lenses or silicone hydrogel contact lenses can be put on directly, because the moisturizing agent of the contact lens packaging solution comprises a salt of hyaluronic acid and a hydrolyzed hyaluronic acid derivative, the packaging solution adsorbed on the soft contact lens can provide sufficient hydration to the contact lenses and wearer initial comfort. Moreover, the pH of the packaging solution is adequate and the color appearance thereof is less yellowish for the application of the contact lenses packaging.

The present invention will be explained in further detail with reference to the examples. However, the present invention is not limited to these examples.

### Preparation Example 1—Preparation of Buffer Solution I

0.71 g of sodium chloride, 0.043 g of sodium borate and 0.43 g of boric acid was added into 98.607 g of deionized water and evenly stirred to be completely dissolved to prepare buffer solution I.

### Example 1

0.10 g of alkyl glyceryl hydrolyzed hyaluronate (Halorepair® manufactured by Kewpie Co. Japan), 0.10 g of sodium hyaluronate (HA-LQH manufactured by Kewpie Co. Japan) and 0.01 g of alginic acid were added into 99.79 g of buffer solution I and stirred evenly to be completely dissolved. The solution was placed into an autoclave and heated to a temperature of 121° C. for 30 minutes to be sterilized to prepare a packaging solution.

### Example 2

0.05 g of alkyl glyceryl hydrolyzed hyaluronate (commercial name: Halorepair® manufactured by Kewpie Co. Japan), 0.15 g of sodium hyaluronate (commercial name: HA-LQH manufactured by Kewpie Co. Japan) and 0.01 g of alginic acid were added into 99.79 g of buffer solution I and stirred evenly to be completely dissolved. The solution was placed into an autoclave and heated to a temperature of 121° C. for 30 minutes to be sterilized to prepare a packaging solution.

### Example 3

0.15 g of alkyl glyceryl hydrolyzed hyaluronate (Halorepair®, manufactured by Kewpie Co. Japan), 0.10 g of sodium hyaluronate (HA-LQH, manufactured by Kewpie Co. Japan) and 0.01 g alginic acid were added into 99.74 g of buffer solution I and stirred evenly to be completely dissolved. The solution was placed into an autoclave and heated to a temperature of 121° C. for 30 minutes to be sterilized to prepare a packaging solution.

### Example 4

0.20 g of alkyl glyceryl hydrolyzed hyaluronate (Halorepair® manufactured by Kewpie Co. Japan), 0.10 g sodium hyaluronate (HA-LQH manufactured by Kewpie Co. Japan) and 0.01 g of alginic acid were added into 99.69 g of buffer solution I and stirred evenly to be completely dissolved. The solution was placed into an autoclave and heated to a temperature of 121° C. for 30 minutes to be sterilized to prepare a packaging solution.

### Comparative Example 1

0.10 g of sodium hyaluronate (HA-LQH) and 0.01 g of alginic acid were added into 99.89 g of buffer solution I and stirred evenly to be completely dissolved. The solution was placed into an autoclave and heated to a temperature of 121° C. for 30 minutes to be sterilized to prepare a packaging solution.

### Comparative Example 2

0.30 g of alkyl glyceryl hydrolyzed hyaluronate (Halorepair®) and 0.01 g of alginic acid were added into 99.69 g of buffer solution I and stirred evenly to be completely dissolved. The solution was placed into an autoclave and heated to a temperature of 121° C. for 30 minutes to be sterilized to prepare a packaging solution.

The contact lens packaging solutions prepared in and obtained from Examples 1 to 4 and Comparative Examples 1 and 2 were evaluated in accordance with the evaluation method described as below. The results obtained were listed in Table 1.

### Viscosity Measurement

The viscosity of a 6.7 ml of packaging solution sample of the contact lens packaging solutions obtained from Examples 1 to 4 and Comparative Examples 1 and 2 was measure at a temperature of 25° C. by a viscometer (Brookfield LVDV-E, manufactured by BROOKFIELD ENGINEERING LAB., INC., US). The test results were listed in Table 1.

### Contact Angle Measurement

The contact lens (silicone hydrogel contact lens manufacture by BenQ Materials Corp, Taiwan) packaged by the contact lens packaging solutions obtained from Examples 1 to 4 and Comparative Examples 1 to 2 was taken out of the package case and wiped by cloth to remove liquid on the lens. The lens was set on a sample stage of the contact angle measuring apparatus (DGD Fast/60 Contact Angle Meter, manufactured by GBX Scientific Ltd., Ireland) and a drop was water was dropped on the lens. The contact angle between the lens surface and the water droplet was automatically measured and calculated. The test results were listed in Table 1.

The evaluation for the average break-up time of water layer on the contact lens

The contact lenses (silicon hydrogel contact lenses, manufactured by BenQ Materials Corp.) packaged by contact lens packaging solution obtained from Examples 1 to 4 and Comparative Examples 1 to 2 were taken out from the packaging container, placed on a semi-spherical surface for 1 minutes and then, to evaluation the break-up time of the water layer in vitro by corneal topographer (OCULUS Keratograph 5M Topographer, manufactured by OCULUS Optikgerate GmbH, Germany). Furthermore, the contact lenses (silicon hydrogel contact lenses, manufactured by BenQ Materials Corp.) packaged by contact lens packaging solution obtained from Examples 1 to 4 and Comparative Examples 1 to 2 were taken out from the packaging container and were worn straight by the test users for 10 to 15 minutes. After wearing 10 to 15 minutes, the test users were asked to open eyes for evaluating the break-up time of the water layer by corneal topographer (OCULUS Keratograph 5M Topographer). The time obtained was the break-up time in vivo. The test results were listed in Table 1.

### Moisture Evaporation Rate

The contact lenses were immersed in the contact lens packaging solution obtained from Examples 1 to 4 and Comparative Examples 1 to 2 for 24 hours, taken out and wiped the residue liquid, places the lenses on the mass balance, weighted and recorded the weight change every minute until 28 minutes. The test results were listed in Table 1.

### pH Value and Osmolality Measurement

5 ml of the contact lens packaging solutions obtained from Examples 1 to 4 and Comparative Examples 1 to 2 was sampled to measure the pH value by pH meter (5220, manufactured by METTLER TOLEDO Copr., Switzerland). 200 l was sampled and measured the osmolality by the Osmometer (3250, manufactured by ADVANCED INSTRUMENTS, LLC, US). The test results were listed in Table 1.

### Color Appearance

The color appearance of the contact lens packaging solutions obtained from Examples 1 to 4 and Comparative Examples 1 to 2 was observed visually.

TABLE 1

| | | | | | Comparative | Comparative |
|---|---|---|---|---|---|---|
| | Example 1 | Example 2 | Example 3 | Example 4 | Example 1 | Example 2 |
| Viscosity | 9.36 | 12.61 | 14.62 | 13.11 | 6.22 | 3.17 |
| Contact angle | 20.03 | 20.67 | 19.30 | 19.37 | 20.00 | 20.67 |
| Water layer break- | 77.38 | 72.27 | 78.94 | 80.60 | 67.07 | 80.41 |
| up time in vitro (s) | | | | | | |
| Water layer break- | 12.04 | 8.91 | 11.87 | 13.53 | 7.43 | 13.55 |
| up time in vivo (s) | | | | | | |
| Moisture | −0.37 | −0.44 | −0.43 | −0.36 | −0.49 | −0.37 |
| evaporation rate | | | | | | |
| (28 min) | | | | | | |
| pH value | 7.15 | 7.23 | 7.07 | 6.90 | 7.32 | 6.75 |
| Osmolality | 315 | 312 | 313 | 309 | 312 | 307 |
| Color appearance | Pale yellow | Clear | Pale yellow | Light yellow | Clear | Yellow |

From the Table 1, the pH values of the contact lens packaging solutions obtained from Examples 1 to 4 are in the range of 6.8 to 7.5 and the osmolality thereof greater than 300 mOsmol/Kg. Moreover, the break-up times of the water layers in vitro of the contact lens packaging solutions obtained from Examples 1 to 4 are all more than 72 seconds, and the break-up times of the water layers in vitro of the contact lens packaging solutions obtained from Examples 1 to 4 are all more than 8 seconds. Thus, after immersing in the present contact lens packaging solution, the contact lenses can provide sufficient hydration for initial wear comfort and alleviated the eye dryness caused by wearing contact lenses. The packaging solution obtained in Comparative Example 1 can not effectively maintain the hydration due to the short water-layer break-up time. The packaging solution obtained in Comparative Example 2 with a low pH value and a yellow appearance after high temperature sterilization is not suitable to be a contact lens packaging solution for high temperature sterilization.

Although particular embodiments have been shown and described, it should be understood that the above discussion is not intended to limit the present invention to these embodiments. Persons skilled in the art will understand that various changes and modifications may be made without departing from the scope of the present invention as literally and equivalently covered by the following claims.

1. A contact lens packaging solution, comprising a buffer solution and 0.1 wt % to 0.4 wt % of a moisturizing agent, wherein the moisturizing agent comprises a salt of hyaluronic acid and a hydrolyzed hyaluronic acid derivative, and a weight ratio of the salt of hyaluronic acid and the hydrolyzed hyaluronic acid derivative is in the range of 1:4 to 4:1.

2. The contact lens packaging solution as claimed in claim 1, wherein a pH value of the contact lens packaging solution is in the range of 6.8 to 7.5.

3. The contact lens packaging solution as claimed in claim 1, wherein the salt of hyaluronic acid in the moisturizing agent is a sodium hyaluronate with a molecular weight in the range of 850,000 to 2,200,000.

4. The contact lens packaging solution as claimed in claim 1, wherein the hydrolyzed hyaluronic acid derivative in the moisturizing agent is an alkyl glyceryl hydrolyzed hyaluronate having a molecular weight less than 10,000 and having a chemical structure of the following formula (I):

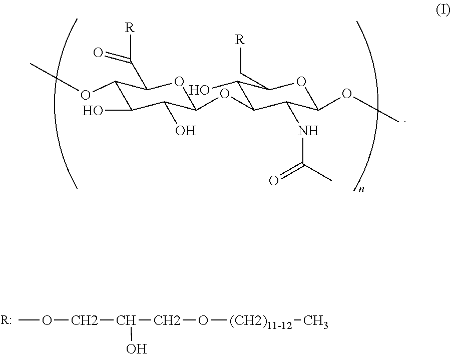

5. The contact lens packaging solution as claimed in claim 1, wherein the buffer solution is 0.1 wt % to 3 wt % of a borate buffer solution or a phosphate buffer solution.

6. The contact lens packaging solution as claimed in claim 1, wherein the buffer solution is a borate buffer solution comprising sodium chloride, boric acid and sodium borate.

7. The contact lens packaging solution as claimed in claim 1, further comprising 0.005 wt % to 0.05 wt % of an alginic acid.

8. The contact lens packaging solution as claimed in claim 1, further comprising an antimicrobial agent, a thickener, a lubricant or a combination thereof.

9. A contact lenses product, comprising a packaging solution and a contact lens immersed in packaging solution, wherein the packaging solution comprises a buffer solution and 0.1 wt % to 0.4 wt % of a moisturizing agent, wherein the moisturizing agent comprises a salt of hyaluronic acid and a hydrolyzed hyaluronic acid derivative, and a weight ratio of the salt of hyaluronic acid and the hydrolyzed hyaluronic acid derivative is in the range of 1:4 to 4:1.

10. The contact lens product as claimed in claim 9, wherein the contact lenses is hydrogel contact lenses or silicone hydrogel contact lenses.
